(19) United States
(12) Patent Application Publication
(10) Pub. No.: US 2025/0251075 A1
(43) Pub. Date: Aug. 7, 2025

(54) MOUNTING ASSEMBLY

(71) Applicant: TECHNOLOGIES CGC INC., Quebec (CA)

(72) Inventors: Martin GAGNE, Quebec (CA); Bruno DESAULNIERS, Quebec (CA); Maude LAUZON, Quebec (CA); Richard LABBE, Quebec (CA); Carl BOUCHARD, Quebec (CA)

(21) Appl. No.: 18/841,814

(22) PCT Filed: Mar. 14, 2023
(86) PCT No.: PCT/CA2023/050325

§ 371 Date: Aug. 27, 2024

Related U.S. Application Data
(60) Provisional application No. 63/319,945, filed on Mar. 15, 2022.

(51) Int. Cl. F16M 11/04; F16M 11/06

(52) U.S. Cl. CPC F16M 11/046; F16M 11/06; F16M 2200/025; F16M 2200/066

(57) ABSTRACT

A mounting assembly includes a track and a fixing assembly. The fixing assembly, which includes outer, positioning and retaining members, can be selectively connected and disconnected from the track, which has a slot defined in an elongate body. When connected to the track, the fixing assembly is moveable longitudinally in the slot. The positioning member, which is at least partially receivable in the slot, is engageable to the elongate body for longitudinally fixing the fixing assembly relative to the track. The retaining member, which is selectively laterally extendable from the positioning member, is movable between a retaining position in which, when the positioning member is at least partially received in the slot, the retaining member engages with the track to retain connection between the fixing assembly and the track, and a release position in which the fixing assembly can be disconnected from the track. Other mounting assemblies are disclosed.

### TECHNICAL FIELD

The present technology relates to mounting assemblies.

### BACKGROUND

Mounting assemblies are used to mount mobile equipment such as medical equipment or electronic equipment to support structures such as walls and/or poles.

Conventionally, mounting mobile equipment to support structures in a secure manner can be a relatively long and difficult process, and may require the use of two or more people and/or the use of both hands. Additionally, conventional mounting assemblies may require mobile equipment be placed at specific positions to be connected and disconnected, which can make it more difficult to mount the mobile equipment to the support structure. Furthermore, particularly when mounting assemblies are used for medical uses, there may be a need to rapidly move a position of the mobile equipment during its use on a patient or in situations when the patient must be moved. Conventional mounting assemblies may not offer such flexibility whilst ensuring the secure attachment of the mobile equipment to the support structure.

Therefore, there is a desire for a mounting assembly that can overcome at least some of the above-described drawbacks.

### SUMMARY

It is an object of the present technology to ameliorate at least some of the inconveniences present in the prior art.

According to one aspect of the present technology, there is provided a . . . .

In some embodiments, a mounting assembly including a track and a fixing assembly. The track includes an elongate body having a longitudinal axis, and a slot defined in the elongate body and extending longitudinally. The fixing assembly can be selectively connected and disconnected from the track in a direction perpendicular to the longitudinal axis. When connected to the track, the fixing assembly is moveable longitudinally in the slot in an unlocked connected mode, and is lockable in the slot in a locked connected mode. The fixing assembly includes an outer member, a positioning member and a retaining member. The positioning member, which is operatively connected to the outer member, has a distal portion receivable in the slot. The positioning member is movable, relative to the outer member, between an extended and a contracted position. In the extended position, in the locked connected mode, the distal portion is engaged with the elongate body of the track when the distal portion of the positioning member is received in the slot for longitudinally fixing the fixing assembly relative to the track. In the contracted position, in the unlocked connected mode, the distal portion is disengaged with the elongate body of the track when the distal portion of the positioning member is received in the slot. The retaining member is selectively extendable from the distal portion of the positioning member, the retaining member being movable between a retaining position and a release position. In the retaining position, when the distal portion of the positioning member is received in the slot, the retaining member engages with the track for retaining connection between the fixing assembly and the track. In the retaining position, the fixing assembly can be disconnected from the track.

In some embodiments, the retaining member includes a pair of arms such that, when the distal portion of the positioning member is received in the slot, in the retaining position, each arm of the pair of arms extends laterally outwardly from the distal portion of the positioning member and delimits movement of the fixing assembly perpendicularly away from the longitudinal axis of the track to retain the connection between the fixing assembly and the track, and in the release position, each arm of the pair of arms is retracted towards the distal portion of the positioning member to permit movement of the fixing assembly perpendicularly away from the longitudinal axis of the track to disconnect the fixing assembly from the track.

In some embodiments, each arm of the pair of arms extends through a wall of the distal portion of the positioning member, the retaining member further comprising a locking member disposed between the pair of arms, the locking member configured to selectively modulate a separation distance between the pair of arms, such that the separation distance of the pair of arms when the pair of arms are in the retaining position is larger than the separation distance when the pair of arms is in the release position.

In some embodiments, the locking member comprises a shaft extending parallel to the longitudinal axis when the distal portion of the positioning member is received in the slot, the shaft having a cross-sectional profile with a variable diameter, such that the separation distance between the pair of arms can be modulated by moving the shaft.

In some embodiments, the locking member has a limiting portion for limiting movement of the shaft.

In some embodiments, the shaft is rotatable and the separation distance between the arms of the pair of arms can be modulated by rotating the shaft.

In some embodiments, the shaft is moveable along a longitudinal axis of the shaft and the separation distance between the arms of the pair of arms can be modulated by translating the shaft along the longitudinal axis.

In some embodiments, the pair of arms are resiliently biased towards each other by a resilient member.

In some embodiments, the pair of arms is a first pair of arms, and the retaining member comprises a second pair of arms spaced longitudinally from the first pair of arms.

In some embodiments, the retaining member comprises a third pair of arms, and the first pair of arms, the second pair of arms and the third pair of arms are spaced longitudinally from one another.

In some embodiments, the outer member includes an inner body, and the locking member is received at least partially within the inner body.

In some embodiments, the outer member includes an actuator which is moveably connected to a proximal portion of the positioning member, the actuator being moveable between an actuated position and a non-actuated position to selectively move the positioning member between the contracted position and the extended position, respectively.

In some embodiments, the actuator is biased toward the non-actuated position.

In some embodiments, the actuator is biased by a resilient member.

In some embodiments, a direction of movement of the actuator between the actuated position and the non-actuated position is perpendicular to a direction of movement of the positioning member between the contracted position and the extended position.

In some embodiments, the positioning member moves between the extended and contracted positions perpendicularly to the longitudinal axis, along a positioning plane containing the longitudinal axis.

In some embodiments, the positioning member includes a first connector, the track includes a plurality of second connectors longitudinally spaced from one another, and wherein the first connector connects to at least one of the plurality of second connectors when the fixing assembly is in the locked connected mode and the positioning member is in the extended position.

In some embodiments, each one of the plurality of second connectors comprises a protrusion, and the first connector is a recess configured to receive the at least one of the plurality of second connector.

In some embodiments, the first connector and the plurality of second connectors each have an arcuate profile.

In some embodiments, the first connector is an upper first connector and a lower first connector, and wherein the upper first connector is configured to connect to one of the plurality of second connectors and the lower first connector is configured to connect to an other one of the plurality of second connectors.

In some embodiments, the track includes a first insert having the plurality of second connectors, the first insert being replaceable with a second insert.

In some embodiments, the slot is a first slot on a first side of the elongate body, the elongate body has a second side opposite to the first side, the second side defining a second slot extending longitudinally along the second side, the second slot having a plurality of third connectors spaced from one another along the second side, the third connectors being configured to connect to the first connector, and the fixing assembly is selectively connectable to the track at either one of the first and second sides.

In some embodiments, the elongate body further has a third side defining a third slot extending longitudinally along the third side, the third slot having a plurality of fourth connectors spaced from one another along the third side, the fourth connectors being configured to connect to the first connector, a fourth side opposite to the third side, the fourth side defining a fourth slot extending longitudinally along the fourth side, the fourth slot having a plurality of fifth connectors spaced from one another along the fourth side, the fifth connectors being configured to connect to the first connector, and the fixing assembly is selectively connectable to the track at either one of the first, second, third and fourth sides.

In some embodiments, the fixing assembly is configured to connect mobile equipment to the track.

According to another aspect of the present technology, there is provided a mounting assembly including a track and a fixing assembly. The track includes an elongate body having a longitudinal axis, and a slot defined in the elongate body and extending longitudinally. The fixing assembly selectively connectable and disconnectable from the track in a direction perpendicular to the longitudinal axis. When connected to the track, the fixing assembly is moveable longitudinally in the slot. The fixing assembly includes an outer member, a positioning member and a retaining member. The positioning member is operatively connected to the outer member. The positioning member is at least partially receivable in the slot and is engageable to the elongate body for longitudinally fixing the fixing assembly relative to the track. The retaining member is selectively laterally extendable from the positioning member. The retaining member is movable between a retaining position in which, when the positioning member is at least partially received in the slot, the retaining member engages with the track to retain connection between the fixing assembly and the track, and a release position in which the fixing assembly can be disconnected from the track.

In some embodiments, the retaining member includes a pair of arms such that, when the positioning member is at least partially received in the slot, in the retaining position, each arm of the pair of arms extends laterally outwardly from the positioning member and delimits movement of the fixing assembly perpendicularly away from the longitudinal axis of the track to retain the connection between the fixing assembly and the track, and in the release position, each arm of the pair of arms is retracted towards the positioning member to permit movement of the fixing assembly perpendicularly away from the longitudinal axis of the track to disconnect the positioning member from the track.

In some embodiments, each arm of the pair of arms extends through a wall of the positioning member, the retaining member further comprising a locking member disposed between the pair of arms, the locking member configured to selectively modulate a separation distance between the pair of arms, such that the separation distance of the pair of arms when the pair of arms are in the retaining position is larger than the separation distance when the pair of arms is in the release position.

In some embodiments, the locking member comprises a shaft extending parallel to the longitudinal axis when the positioning member is at least partially received in the slot, the shaft having a cross-sectional profile with a variable diameter, such that the separation distance between the pair of arms can be modulated by moving the shaft.

In some embodiments, the locking member has a limiting portion for limiting movement of the shaft.

In some embodiments, the shaft is rotatable and the separation distance between the arms of the pair of arms can be modulated by rotating the shaft.

In some embodiments, the shaft is moveable along a longitudinal axis of the shaft and the separation distance between the arms of the pair of arms can be modulated by translating the shaft along the longitudinal axis.

In some embodiments, the pair of arms are resiliently biased towards each other by a resilient member.

In some embodiments, the pair of arms is a first pair of arms, and the retaining member comprises a second pair of arms spaced longitudinally from the first pair of arms.

In some embodiments, the retaining member comprises a third pair of arms, and the first pair of arms, the second pair of arms and the third pair of arms are spaced longitudinally from one another.

In some embodiments, the outer member includes an inner body, and the locking member is received at least partially within the inner body.

In some embodiments, the fixing assembly is configured to connect mobile equipment to the track.

In some embodiments, the slot is a first slot on a first side of the elongate body, the elongate body has a second side opposite to the first side, the second side defining a second slot extending longitudinally along the second side, the second slot having a plurality of third connectors spaced from one another along the second side, the third connectors being configured to connect to the first connector, and the fixing assembly is selectively connectable to the track at either one of the first and second sides.

In some embodiments, the elongate body further has a third side defining a third slot extending longitudinally along the third side, the third slot having a plurality of fourth connectors spaced from one another along the third side, the fourth connectors being configured to connect to the first connector, a fourth side opposite to the third side, the fourth side defining a fourth slot extending longitudinally along the fourth side, the fourth slot having a plurality of fifth connectors spaced from one another along the fourth side, the fifth connectors being configured to connect to the first connector, and the fixing assembly is selectively connectable to the track at either one of the first, second, third and fourth sides.

According to another aspect of the present technology, there is provided a mounting assembly including a track and a fixing assembly. The track includes an elongate body having a longitudinal axis, and a slot defined in the elongate body and extending longitudinally. The fixing assembly which is connectable to the track includes an outer member and a positioning member. The outer member includes an actuator. The positioning member, which is operatively connected to the outer member, has a proximal portion and a distal portion receivable in the slot. The positioning member is movable, relative to the outer member, between an extended position, in which when the distal portion is received in the slot, the distal portion engages the elongate body of the track for longitudinally fixing the fixing assembly relative to the track, and a contracted position, in which when the distal portion is received in the slot, the distal portion is disengaged from the elongate body of the track for permitting longitudinal movement of the fixing assembly relative to the track. The actuator is moveably connected to the proximal portion of the positioning member, and is selectively moveable between an actuated position to move the positioning member to the contracted position, and a non-actuated position in which the positioning member is in the extended position.

In some embodiments, the actuator is biased toward the non-actuated position.

In some embodiments, the actuator is biased by a resilient member.

In some embodiments, a direction of movement of the actuator between the actuated position and the non-actuated position is perpendicular to a direction of movement of the positioning member between the contracted position and the extended position.

In some embodiments, the positioning member moves between the extended and contracted positions perpendicularly to the longitudinal axis, along a positioning plane containing the longitudinal axis.

In some embodiments, the positioning member includes a first connector on the distal portion, the elongate track includes a plurality of second connectors longitudinally spaced from one another, and wherein the first connector connects to one of the plurality of second connectors when the positioning member is in the extended position.

In some embodiments, each one of the plurality of second connectors comprises a protrusion, and the first connector is a recess configured to receive one of the plurality of second connector.

In some embodiments, the first connector and the plurality of second connectors each have an arcuate profile.

In some embodiments, the first connector is an upper first connector and a lower first connector, and wherein the upper first connector is configured to connect to one of the plurality of second connectors and the lower first connector is configured to connect to an other one of the plurality of second connectors.

In some embodiments, the track includes a first insert having the plurality of second connectors, the first insert being replaceable with a second insert.

In some embodiments, the slot is a first slot on a first side of the elongate body, the elongate body has a second side opposite to the first side, the second side defining a second slot extending longitudinally along the second side, the second slot having a plurality of third connectors spaced from one another along the second side, the third connectors being configured to connect to the first connector, and the fixing assembly is selectively connectable to the track at either one of the first and second sides.

In some embodiments, the elongate body further has a third side defining a third slot extending longitudinally along the third side, the third slot having a plurality of fourth connectors spaced from one another along the third side, the fourth connectors being configured to connect to the first connector, a fourth side opposite to the third side, the fourth side defining a fourth slot extending longitudinally along the fourth side, the fourth slot having a plurality of fifth connectors spaced from one another along the fourth side, the fifth connectors being configured to connect to the first connector, and the fixing assembly is selectively connectable to the track at either one of the first, second, third and fourth sides.

In some embodiments, the fixing assembly is configured to connect mobile equipment to the track.

According to another aspect of the present technology, there is provided a fixing assembly selectively connectable to a track, the fixing assembly including an outer member, a positioning member and a retaining member. The positioning member, which is operatively connected to the outer member, has a distal portion configured to be receivable in the slot. The positioning member is movable between an extended position and a contracted position. A distance from the outer member to the distal portion is greater when the positioning member is in the extended position than when the positioning member is in the contracted position. The retaining member is selectively extendable from the distal portion of the positioning member. The retaining member is movable between a retaining position in which the retaining member extends laterally from the distal portion of the positioning member and a release position in which the retaining member is retracted towards the distal portion of the positioning member.

According to another aspect of the present technology, there is provided a fixing assembly selectively connectable to a track. The fixing assembly includes an outer member, a positioning member, a retaining member and a locking member. The positioning member, which is operatively connected to the outer member, has at least one positioning connector configured to connect to a connector of the track, wherein the positioning member is movable between an extended position and a contracted position. The retaining member, which is operatively connected to the positioning member, is movable between a retaining position and a release position. The locking member is operatively connected to the positioning member and engaged to the retaining member. The locking member is adjustable between an unlocked position and a locked position. In the unlocked position, the locking member engages the retaining member to move the retaining member to the release position, and in the locked position, the locking member engages the retaining member to move the retaining member to the retaining position.

### Advantages

In certain embodiments, the mounting assembly is configured to mount mobile equipment such as fragile apparatus, medical equipment and/or electronic equipment to a support structure. The support structure may comprise one or more of a wall, a ceiling or a floor of a transport vehicle or a building. The support structure may also comprise rails or poles within the transport vehicle or the building, or items such as a stretcher, a wheelchair and/or a moveable base.

Embodiments of the mounting assembly enable the securing of the mobile equipment to the support structure during transportation or other multi-directional movement. Transportation could be off-road and jarring. In certain embodiments, the mobile equipment can remain secured to the support structure during high acceleration and deceleration events, as well as travel on uneven surfaces. In certain embodiments, the mounting assembly can withstand impacts of up to 30G.

Embodiments of the mounting assembly enable the connection and the disconnection of the mobile equipment by a single person, and do not require more than one person. In some instances, the connection and disconnection can be done by a single hand. In some other instances, the connection and disconnection can be done manually, without the use of tools.

Embodiments of the mounting assembly enable the unconstrained use of the mobile equipment during transportation by allowing access to required parts of the mobile equipment, and not obscuring or blocking those parts.

Embodiments of the mounting enable the securing of mobile equipment of different sizes, shapes and configurations to support structures.

Embodiments of the mounting assembly enable the mobile equipment to be easily connected and disconnected to the support structure by a single hand.

Embodiments of the mounting assembly enable the mobile equipment to be easily adjusted in vertical and/or horizontal direction without necessarily requiring the mobile equipment to be disconnected.

In the context of the present specification, unless expressly provided otherwise, the words “first”, “second”, “third”, etc. have been used as adjectives only for the purpose of allowing for distinction between the nouns that they modify from one another, and not for the purpose of describing any particular relationship between those nouns.

It must be noted that, as used in this specification and the appended claims, the singular form “a”, “an” and “the” include plural referents unless the context clearly dictates otherwise.

As used herein, the term “about” in the context of a given value or range refers to a value or range that is within 20%, preferably within 10%, and more preferably within 5% of the given value or range.

As used herein, the term “and/or” is to be taken as specific disclosure of each of the two specified features or components with or without the other. For example “A and/or B” is to be taken as specific disclosure of each of (i) A, (ii) B and (iii) A and B, just as if each is set out individually herein.

Implementations of the present technology each have at least one of the above-mentioned object and/or aspects, but do not necessarily have all of them. It should be understood that some aspects of the present technology that have resulted from attempting to attain the above-mentioned object may not satisfy this object and/or may satisfy other objects not specifically recited herein.

Additional and/or alternative features, aspects, and advantages of implementations of the present technology will become apparent from the following description, the accompanying drawings, and the appended claims.

### BRIEF DESCRIPTION OF THE DRAWINGS

For a better understanding of the present technology, as well as other aspects and further features thereof, reference is made to the following description which is to be used in conjunction with the accompanying drawings, where:

FIG. 1 is a perspective view taken from a front, top, left side of a mounting assembly.

FIG. 2 is a front elevation view of the mounting assembly of FIG. 1.

FIG. 3A is an exploded perspective view taken from a rear, top, right side of a fixing assembly of the mounting assembly of FIG. 1.

FIG. 3B is an exploded perspective view taken from a front, top, right of the fixing assembly of FIG. 3A.

FIG. 4A is a cross-sectional view of the mounting assembly of FIG. 1, with retaining arms of the fixing assembly being in a release position, taken across the lines 4A-4A of FIG. 2.

FIG. 4B is a cross-sectional view of the mounting assembly of FIG. 1, with the retaining arms of the fixing assembly being in a release position, taken across the lines 4B-4B of FIG. 2.

FIG. 4C is a cross-sectional view of the mounting assembly of FIG. 1, with retaining arms of the fixing assembly being in a release position, taken across the lines 4C-4C of FIG. 2.

FIG. 5A is a cross-sectional view of the mounting assembly of FIG. 1, with retaining arms of the fixing assembly being in a retaining position, and a positioning member of the fixing assembly being in a contracted position, taken across the lines 4A-4A of FIG. 2.

FIG. 5B is a cross-sectional view of the mounting assembly of FIG. 1, with retaining arms of the fixing assembly being in a retaining position, and the positioning member being in the contracted position, taken across the lines 4B-4B of FIG. 2.

FIG. 5C is a cross-sectional view of the mounting assembly of FIG. 1, with retaining arms of the fixing assembly being in a retaining position, and the positioning member being in the contracted position, taken across the lines 4C-4C of FIG. 2.

FIG. 6A is a cross-sectional view of the mounting assembly of FIG. 1, with the retaining arms being in a retaining position, and the positioning member being in an extended position, taken across the lines 4A-4A of FIG. 2.

FIG. 6B is a cross-sectional view of the mounting assembly of FIG. 1, with retaining arms of the fixing assembly being in a retaining position, and the positioning member being in the extended position, taken across the lines 4B-4B of FIG. 2.

FIG. 6C is a cross-sectional view of the mounting assembly of FIG. 1, with retaining arms of the fixing assembly being in a retaining position, and the positioning member being in the extended position, taken across the lines 4C-4C of FIG. 2.

FIG. 7A is a perspective view taken from a top, front, right side of the mounting assembly of FIG. 1 connected to a movable base.

FIG. 7B is a perspective view taken from a top, front, right side of the mounting assembly of FIG. 1 connected to support structure.

FIG. 8 is a cross-sectional view of a four-sided track according to an embodiment of the present technology.

### DETAILED DESCRIPTION

The present disclosure is not limited in its application to the details of construction and the arrangement of components set forth in the following description or illustrated in the drawings. The disclosure is capable of other embodiments and of being practiced or of being carried out in various ways. Also, the phraseology and terminology used herein is for the purpose of description and should not be regarded as limiting. The use of “including”, “comprising”, or “having”, “containing”, “involving” and variations thereof herein, is meant to encompass the items listed thereafter as well as, optionally, additional items. In the following description, the same numerical references refer to similar elements.

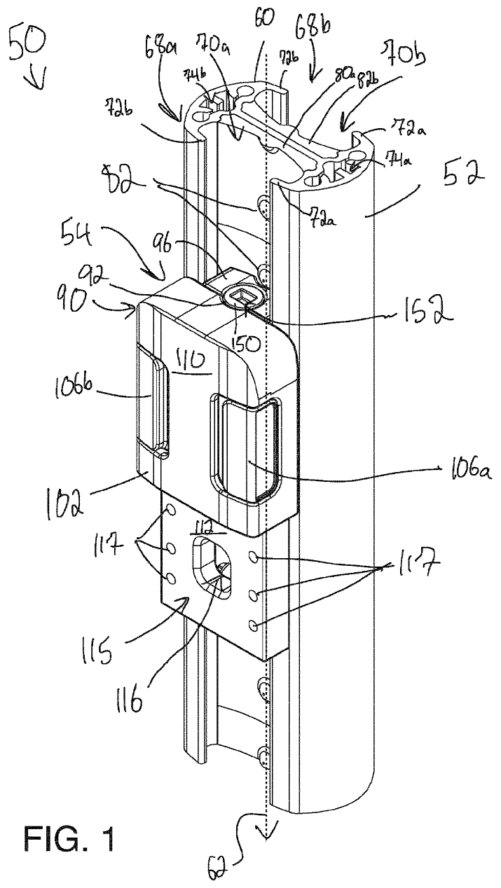

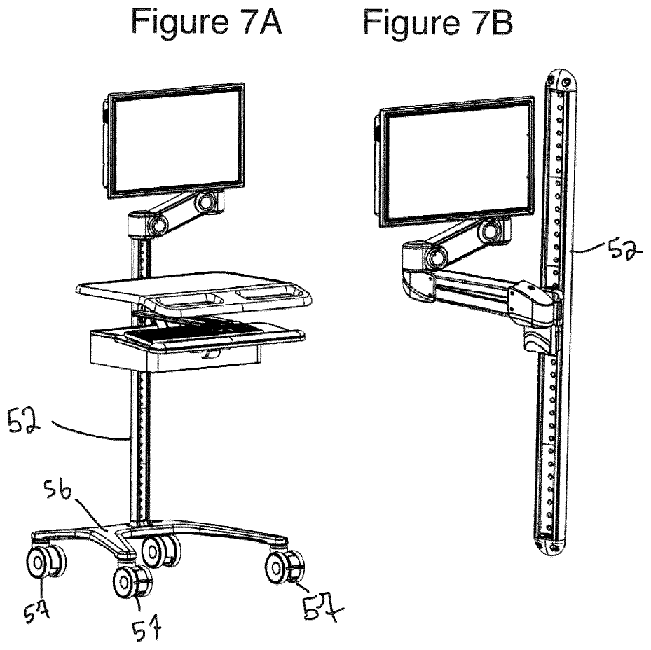

Referring to FIG. 1, there is provided a mounting assembly 50. The mounting assembly 50 includes a track 52 and a fixing assembly 54. As will be described in greater detail herebelow, the fixing assembly 54, which is configured to fix mobile equipment such as medical equipment or electronic equipment to the track 54, is selectively connectable to the track 52. The fixing assembly 54, when connected to the track 52, can be locked relative to the track 52 in a selected position or can slide relative to the track 52 from one position to another position. The fixing assembly 54 can also be disconnected from the track 52 and, can be prevented from being disconnected from the track 52. In some embodiments, as shown in FIG. 7A, the track 52 is connected to a mobile base 56 having wheels 57. In other embodiments, as shown in FIG. 7B, the track 52 is configured to be connected to a support structure and/or a support surface such as a wall, ceiling or floor of a vehicle (e.g. ambulance, helicopter, airplane). In certain embodiments, the locking and/or sliding of the fixing assembly 54 can be performed single-handedly by an operator of the mounting assembly 50.

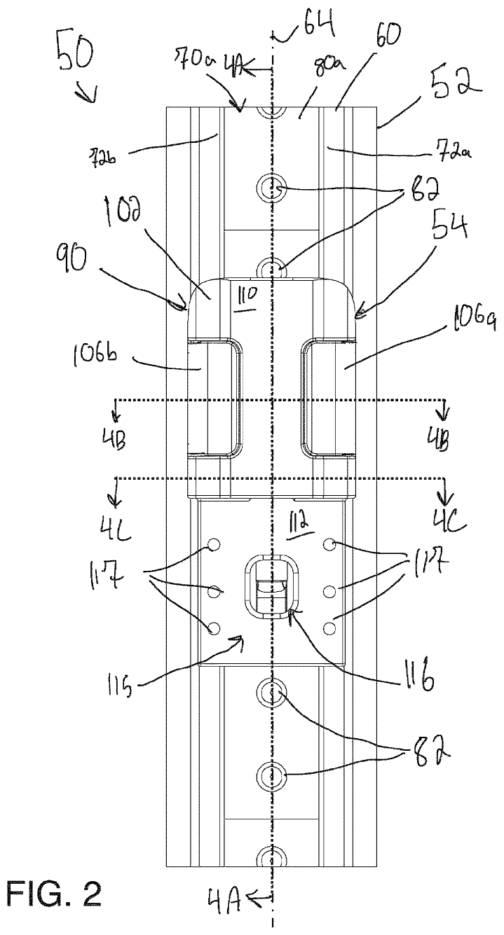

Referring to FIGS. 1 and 2, the track 52 will first be described. The track 52 includes an elongate body 60 that has a longitudinal axis 62. In the present embodiment, the longitudinal axis 62 is illustrated as extending in a generally vertical direction for vertical mounting of the track 52 such as on a moveable base or on a wall as shown in FIGS. 7A and 7B. In other embodiments, the track 52 can be mounted horizontally, such as on a floor or ceiling. In yet other embodiments, the track 52 can be mounted on an inclined surface. The elongate body 60 also has a vertical center plane 64 that contains the longitudinal axis 62. Extending perpendicular to the vertical center plane 64, the elongate body 60 also has a lateral center plane 66. The lateral center plane 66 defines a front side 68a of the elongate body 60 and a rear side 68b of the elongate body 60.

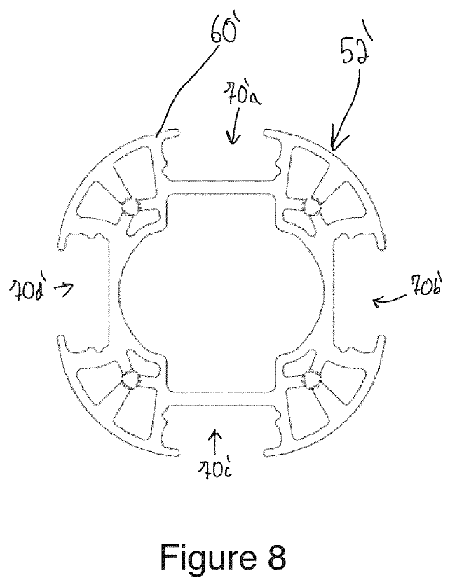

The track 52 has a front slot 70a defined on the front side 68a of the elongate body 60 and a rear slot 70b defined on the rear side 68b of the elongate body 60. The front and rear sides 68a, 68b are opposite to one another. It is contemplated that in some embodiments, the track 52 could only have a single slot. In yet other embodiments, the track 52 could have more than two slots. For instance, referring to FIG. 8, in which is shown an alternate embodiment of the track 52, namely track 52′, the elongate body 60′ has a four-sided cross-sectional profile (where the cross-section is taken along a horizontal plane), such that the track 52′ has four slots 70a′, 70b′, 70c′, 70d′. Referring back to FIGS. 1 and 2, each of the front and rear slots 70a, 70b has left and right retaining portions 72a, 72b. Each of the left and right retaining portions 72a, 72b is a lipped portion of the respective slot. As will be described in greater detail below, the fixing assembly 54 can be selectively connected to the track 52 at either one, or both, of the front and rear slots 70a, 70b.

The elongate body 60 may be partially hollow. In this respect, on each lateral side of the front and rear slots 70a, 70b, the elongate body 60 defines apertures 74a, 74b that extend along the longitudinal axis 62, along an entirety of the elongate body 60. The apertures 74a, 74b can help reduce material required to manufacture the elongate body 60 translating for cost savings in manufacturing as well as shipping. It is understood that the shape of the apertures 74a, 74b could vary from one embodiment to another. In some instances, the apertures 74a, 74b could be omitted altogether.

The track 52 includes a front insert 80a that is received in the front slot 70a, and a rear insert 80b that is received in the rear slot 70b. More precisely, the front and rear inserts 80a, 80b are respectively disposed at a base of the front and rear slots 70a, 70b. The front and rear inserts 80a, 80b are configured so that once they are received in the front and rear slots 70a, 70b, they remain fixed relative to the elongate body 60. It is contemplated that in some embodiments, the inserts 80a, 80b could be omitted.

The track 52 also includes a plurality of track connectors 82, for selective engagement with the fixing assembly 54, that are longitudinally spaced from one another (i.e., spaced along the longitudinal axis 62). The track connectors 82 are protrusions that have an arcuate profile. In some embodiments, the track connectors 82 could be recesses or apertures. In other embodiments, the track connectors 82 could not have an arcuate profile. In other embodiments, the track connectors 82 could be omitted. In the present embodiment, the track connectors 82 are disposed on the inserts 80a, 80b. In some embodiments, for instance when the inserts 80a, 80b are omitted, the track connectors 82 could be disposed on the elongate body 60. For example, the track connectors 82 could be machined on the elongate body 60.

When desired, one or both of the front and rear inserts 80a, 80b can be replaced with another same or different insert. A different insert could be the same, or could differ in various ways. For instance, a different insert could have different types of track connectors 82 thereon (e.g., rectangular protrusions) and/or could have a different spacing between the track connectors 82.

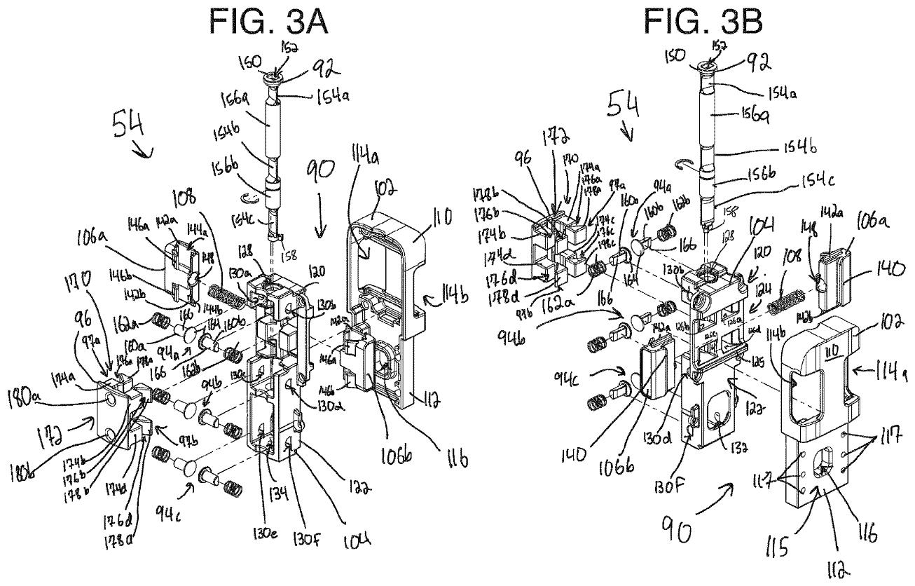

Turning now to FIGS. 3A and 3B, the fixing assembly 54 will be described in greater detail. The fixing assembly 54 includes an outer member 90, a locking member 92, upper, intermediate and lower pairs of retaining arms 94a, 94b, 94c and a positioning member 96.

Starting from a front of the fixing assembly 54, the outer member 90 includes an outer body 102, an inner body 104, left and right actuators 106a, 106b and a spring 108.

The outer body 102, in which a rear side thereof is configured to receive a portion of the inner body 104 therein, has an upper portion 110 and a lower portion 112. The upper portion 110 defines laterally spaced left and right apertures 114a, 114b. As will be described below, the left and right apertures 114a, 114b are configured to, respectively receive portions of the left and right actuators 106a, 106b therein. The lower portion 112 is a connecting interface 115 that defines a central aperture 116 and six connecting apertures 117. The connecting interface 115 is configured to connect to the mobile equipment, such as via an interfacing coupling device as described in WO 2021/062558 filed Oct. 2, 2020, the contents of which are incorporated herein by reference in their entirety. It is contemplated that in some embodiments, the connecting interface 115 could be configured to connect to other devices such as oxygen bottle holders, baskets, drawers, battery packs and/or computer brackets. The interfacing coupling device enables a swift and easy connection between the mobile equipment and the outer member 90.

Turning now to the inner body 104, a portion of which is configured to be received in the rear side of the outer body 102, the inner body 104 has an upper portion 120 and a lower portion 122. The upper portion 120 defines a lateral recess 124 that is configured to receive portions of the left and right actuators 106a, 106b therein. Within the lateral recess 124, the upper portion 120 defines an upper slot (not shown) and a lower slot 125. Also within the lateral recess 124, the upper portion 120 defines four apertures 126a, 126b, 126c, 126d. It is contemplated that in other embodiments, there could be more or less than four apertures. As will be described below, the four apertures 126a, 126b, 126c, 126d are configured to receive portions of the positioning member 96 therein. The upper portion 120 also defines apertures configured to receive fasteners (not shown) to fasten the inner body 104 to the outer body 102. In some embodiments, the outer and inner bodies 102, 104, could be connected differently, such as, for example, with an adhesive. Rearward of the lateral recess 124, and atop of the inner body 104, there is defined a lock aperture 128 that is configured to receive the locking member 92 therein. On lateral sides of the upper portion 120, once again rearward of the lateral recess 124, there is defined upper apertures 130a, 130b that are configured to receive the upper pair of retaining arms 94a.

The lower portion 122 defines a central aperture 132 that, when the inner body 104 is connected to the outer body 102, is aligned with the central aperture 126. On lateral sides of the lower portion 122, there are defined intermediate apertures 130c, 130d. Vertically below the intermediate apertures 130c, 130d, still on the lateral sides of the lower portion 122, there are defined lower apertures 130e, 130f. The intermediate apertures 130c, 130d are configured to receive the intermediate pair of retaining arms 94b, and the lower apertures 130e, 130f, are configured to receive the lower pair of retaining arms 94c.

Still referring to FIGS. 3A and 3B, and now focusing on the left and right actuators 106a, 106b, which, when the outer member 90 is assembled, are disposed between the inner and outer bodies 102, 104. A front portion of each of the left and right actuators 106a, 106b is partially received through, respectively, the left and right apertures 114a, 114b. A rear portion of each of the left and right actuators 106a, 106b is further partially received in the lateral recess 124. The left and right actuators 106a, 106b are mirror versions of each other, and thus only the left actuator 106a will be described in detail herewith.

The left actuator 106a has a front abutting portion 140. The front abutting portion 140 is configured to abut the outer body 102 once the left actuator 106a reaches an actuated position, upon actuation. The left actuator 106a has an upper guiding tab 142a on a top thereof, and lower guiding tab 142b on a bottom thereof. The upper and lower guiding tabs 142a, 142b extend along the left actuator 106a such that when the outer member 90 is assembled and the fixing assembly 54 is connected to the track 52, the upper and lower guiding tabs 142a, 142b are generally parallel to the lateral center plane 66. The upper tab 142a is receivable in the upper slot (not shown) of the inner body 102 and the lower tab 142b is received in the lower slot 125 of the inner body 102. The engagement between the upper and lower tabs 142a, 142b and the upper and lower slots 125 result in a movement of the left actuator 106a being guided in one direction, and restricted in another direction perpendicular thereto.

The left actuator 106a further defines, on its rear side, an upper recess 144a and a lower recess 144b. As will be described in greater detail below, the upper and lower recesses 144a, 144b are configured to receive portions of the positioning member 96 therein. The left actuator 106a has, also on its rear side, an upper guiding segment 146a and a lower guiding segment 146b. The upper and lower guiding segments 146a, 146b project rearward from a body of the left actuator 106a, at an angle therefrom. The upper and lower guiding segments 146a, 146b partially extend over the upper and lower recesses 144a, 144b. Additionally, the left actuator 106a defines, on its lateral side, a spring aperture 148 configured to receive a spring 108.

The spring 108 is received in the spring aperture 148 of the left and right actuators 106a, 106b. Each end of the spring 108 respectively abuts an inner surface of the left and right actuators 106a, 106b. It is contemplated that in other embodiments, the spring 108 could be another resilient member such as an elastic member, a shape memory alloy, or the like.

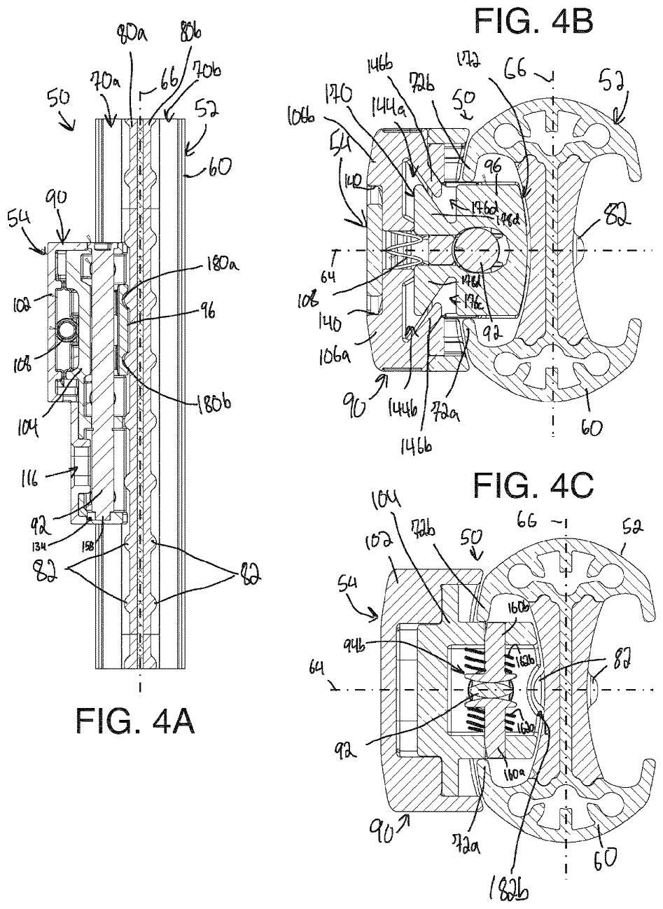

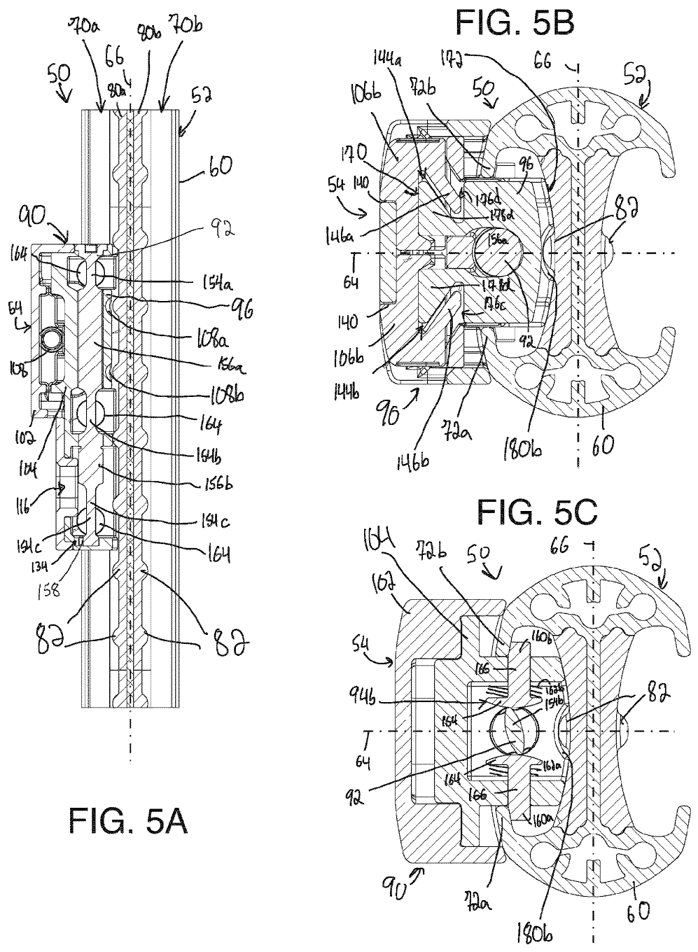

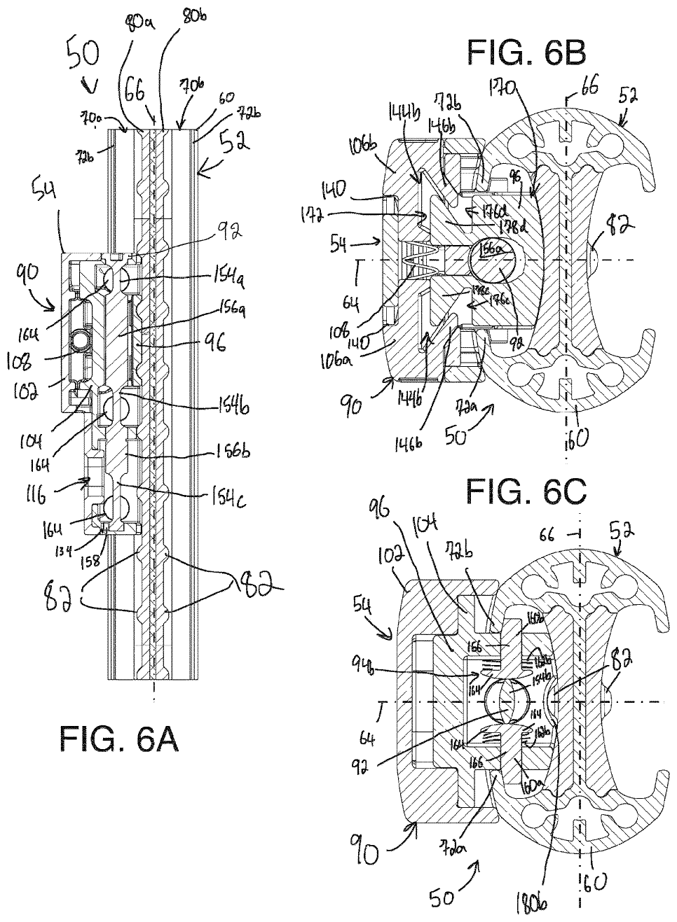

Thus, when the outer member 90 is assembled, the left and right actuators 106a, 106b are moveable between a non-actuated position (FIGS. 4B and 6B) and an actuated position (FIG. 5B). When the fixing assembly 54 is connected to the track 52, the movement of the left and right actuators 106a, 106b between the non-actuated and actuated positions is generally parallel to the lateral center plane 66 (i.e., generally perpendicular to the longitudinal axis 62). The left and right actuators 106a, 106b reach the actuated position when the front abutting portions 140 abut the outer body 102, as shown in FIG. 5B. As the left and right actuators 106a, 106b are moving towards the actuated position, the spring 108 is being resiliently deformed (i.e., compressed). As soon as the spring 108 is resiliently deformed, (i.e., as soon as the left and right actuators are offset from the non-actuated position, the spring 108 biases the left and right actuators 106a, 106b toward the non-actuated position.

With continued reference to FIGS. 3A and 3B, the fixing assembly 54 also includes the locking member 92, which is a rotatable shaft that is sized and configured to be received in the lock aperture 128 of the inner body 104. When the locking member 92 is received in the lock aperture 128, and the fixing assembly 54 is connected to the track 52, the locking member 92 extends parallel to the longitudinal axis 62. Starting from a top of the locking member 92, the locking member 92 has a head portion 150. Below the head portion 150, the locking member 92 has an upper engaging portion 154a, followed by an upper body portion 156a, an intermediate engaging portion 154b, a lower body portion 156a and a lower engaging portion 154c. Below the lower engaging portion 154c, the locking member 92 has a limiting portion 158.

The head portion 150 defines a recess 152. It is contemplated that in some embodiments, the recess 152 could be a protrusion instead. The recess 152 is configured to receive a tool, such as a hex key for example, therein, as will be described in greater detail below, to rotate the locking member 92.

The upper, intermediate, and lower engaging portions 154a, 154b, 154c are configured to engage with, respectively, the upper, intermediate and lower pair of retaining arms 94a, 94b, 94c. Cross-sectional areas of the upper, intermediate, and lower engaging portions 154a, 154b, 154c taken across a horizontal plane are the same, and define a generally oval shape (i.e., one axis of the cross-sections of the upper, intermediate, and lower engaging portions 154a, 154b, 154c is larger than another axis of the cross-sections of the upper, intermediate, and lower engaging portions 154a, 154b, 154c). Thus, cross-sectional area of the locking member 92 has, at the upper, intermediate and lower engaging portions 154a, 154b, 154c a variable diameter along a center of the cross-sectional areas.

The upper and lower body portions 156a, 156b connect the upper, intermediate, and lower engaging portions 154a, 154b, 154c. Cross-sectional areas of the upper and lower body portion 156a, 156b taken across a horizontal plane are the same, and define a generally circular shape.

Thus, the cross-sectional area of the locking member 92 has a variable diameter along its length. Upon comparison, the cross-sectional area of each of the upper and lower body portions 156 are larger than the cross-sectional area of each of the upper, intermediate and lower engaging portions 154a, 154b, 154c. It is contemplated that in some embodiments, the cross-sectional area of each of the upper, intermediate, and lower engaging portions 154a, 154b, 154c could be greater or smaller compared to the cross-sectional area of the upper and lower body portions 156a, 156b.

The limiting portion 158 of the locking member 92 is configured to be received in a recess 134 (best seen in FIG. 3A) that is defined in the inner body 104. The recess 134 is shaped to enable the locking member 92 to have a rotational range of motion of about 100 degrees (i.e., the limiting portion 158 abuts the inner body 104 after rotating 100 degrees). It is contemplated that in other embodiments, the locking member 92 could have a rotational range of motion of more or less than 100 degrees. In the present embodiment, when the locking member 92 abuts one side of the inner body 104, the locking member 92 is in a locking member release position (shown in FIG. 4C), where the larger axis of each of the cross-sections of the upper, intermediate, and lower engaging portions 154a, 154b, 154c is generally parallel to the vertical center plane 64. When the locking member 92 abuts the other side of the inner body 104 (i.e., is rotated 100 degrees), the locking member 92 is in a locking member retaining position (almost shown in FIG. 6C), where a projection of the larger axis of each of the cross-sections of the upper, intermediate, and lower engaging portions 154a, 154b, 154c intersects the lateral center plane 66.

It is contemplated that in some embodiments, the locking member 92 could be a translatable shaft with variable cross-sectional size and/or shape instead of a rotatable shaft.

Still referring to FIGS. 3A and 3B, the fixing assembly 54 also includes the upper, intermediate and lower pair of retaining arms 94a, 94b, 94c. It is contemplated that in some embodiments, there could be more or less than three pairs of retaining arms. The upper, intermediate and lower retaining pair of arms 94a, 94b, 94c are retaining members. It is contemplated that the upper, intermediate and lower pairs of retaining arms 94a, 94b, 94c could come in another form, so long as they extend laterally from the fixing assembly 54.

As the upper, intermediate and lower pair of retaining arms 94a, 94b, 94c are similar, only the upper pair of retaining arms 94a, 94b, 94c will be described in detail herewith.

The upper pair of retaining arms 94 includes left and right retaining arms 160a, 160b as well as left and right springs 162a, 162b. It is contemplated that the left and right spring 162a, 162b could be replaced with other resilient members such as elastic members, shape memory alloys, or the like. The left and right retaining arms 160a, 160b generally have a rivet-like shape, such that each one of the left and right retaining arms 160a, 160b has a head portion 164 and a body portion 166 extending from the head portion 164. In the present embodiment, the body portion 166 of the left retaining arm 160a is configured to extend opposite to the body portion 166 of the right retaining arm 160b, such that the head portions 164 of the left and right retaining arms 160a, 160b are oriented towards one another. The body portion 166 of the left retaining arm 160a is received in the left spring 162a, and a section of the body portion 166 is received through the upper aperture 130a. Similarly, the body portion 166 of the right retaining arm 160b is received in the right spring 162b, and a section of the body portion 166 is received through the upper aperture 130b.

Thus, when the fixing assembly 54 is assembled and best seen in FIGS. 4C, 5C and 6C, one end of the left spring 162a abuts the head portion 164 of the left retaining arm 160a, and the other end of the left spring 162a abuts an interior surface of the inner body 104. Similarly, one end of the right spring 162b abuts the head portion 164 of the right retaining arm 160b, and the other end of the right spring 162b abuts an interior surface of the inner body 104.

The left and right retaining arms 160a, 160b are movable between a release position (FIG. 4C) and a retaining position (FIGS. 5C and 6C). A separation distance measured between the head portions 164 of the left and right retaining arms 160a, 160b is larger when the left and right retaining arms 160a, 160b are in the retaining position than when the left and right retaining arms 160a, 160b are in the release position. The left and right springs 162a, 162b are resiliently deformed (i.e., compressed) when the left and right retaining arms 160a, 160b are in the retaining position, such that the left and right springs 162a, 162b bias the left and right retaining arms 160a, 160b toward the release position. In other words, the left and right springs 162a, 162b bias the left and right retaining arms 160a, 160b towards one another such that the left and right retaining arms 160a, 160b compress the locking member 92. As will be described in greater detail below, the left and right retaining arms 160a, 160b, applying a force to the locking member 92 can help prevent the rotational movement of the locking member 92. When the fixing assembly 54 is connected to the track 52, the left and right retaining arms 160a, 160b move between the release and retaining positions generally laterally (i.e., parallel to the lateral center plane 66 and perpendicular to the longitudinal axis 62). As will be described in greater detail below, the upper engaging portion 156a is configured to engage with the left and right retaining arms 160a, 160b to modulate the position of the left and right retaining arms 160a, 160b between the release and retaining positions.

Still referring to FIGS. 3A and 3B, the positioning member 96, which is operatively connected to the outer member 90, will now be described in greater detail. The positioning member 96 has an upper unit 97a and a lower unit 97b. The upper and lower units 97a, 97b, which are parallel to one another, are integral. It is contemplated that in some embodiments, the positioning member 96 could only have one unit, or could have three or more units. Each of the upper and lower units 97a, 97b has a proximal portion 170 and a distal portion 172, where the proximal portion 170 is forward from the distal portion 172. The proximal portion 170 of the upper unit 97a includes an upper left segment 174a, an upper right segment 174b, and the proximal portion 170 of the lower unit 97b includes a lower left segment 174c and a lower right segment 174d. It is contemplated that in other embodiments, the positioning member 96 could include more or less than four segments, for instance when there are there three units or when there is a single unit.

The upper and lower left segments 174a, 174c are respectively laterally spaced from the upper and lower right segments 174b, 174d so that upon assembly of the fixing assembly 54, the locking member 92 can be received between the upper left and right segments 174a, 174b and between the lower left and right segments 174c, 174d. Additionally, the upper left and right segments 174a, 174b are respectively vertically spaced from the lower left and right segments 174c, 174d. The upper and lower left and right segments 174a, 174b, 174c, 174d are configured to be respectively received in the apertures 126a, 126b, 126c, 126d defined in the inner body 104.

The upper left segment 174a defines a triangular recess 176a configured to receive the upper guiding segment 146a of the left actuator 106a. The upper left segment 174a also has an actuator engaging portion 178a configured to be received in the upper recess 144a of the left actuator 106a.

The upper right segment 174b defines a triangular recess 176b configured to receive the upper guiding segment 146a of the right actuator 106b. The upper right segment 174b also has an actuator engaging portion 178b configured to be received in the upper recess 144a of the right actuator 106b.

The lower left segment 174c defines a triangular recess 176c configured to receive the lower guiding segment 146b of the left actuator 106a. The lower left segment 174c also has an actuator engaging portion 178c configured to be received in the lower recess 144b of the left actuator 106a.

The lower right segment 174d defines a triangular recess 176d configured to receive the lower guiding segment 146b of the right actuator 106b. The lower right segment 174d also has an actuator engaging portion 178d configured to be received in the lower recess 144b of the right actuator 106b.

The triangular recesses 176a, 176b, 176c, 176d and the actuator engaging portions 178a, 178b, 178c, 178d, in part due to their shape, as will be described in greater detail below, enable a movement of the positioning member 96 generally perpendicular to the movement of the actuators 106a, 106b, and stop the movement of the positioning member 96 relative to the actuators 106a, 106b by abutment upon reaching a given position.

Turning now to the distal portions 172 of the upper and lower units 97a, 97b, which are receivable in either one of the slots 70a, 70b, the positioning member 96 has an upper positioning connector 180a, and a lower positioning connector 180b. The upper and lower positioning connectors 180a, 180b are vertically spaced (i.e., spaced along the longitudinal axis 62 when the fixing assembly 54 is connected to the track 52). More precisely, the upper unit 97a has the upper positioning connector 180a, and the lower unit 97b has the lower positioning connector 180b. It is contemplated that in some embodiments, the positioning member 96 could have a single positioning connector (e.g., when there is a single unit) or three or more positioning connectors (e.g., when there are three or more units). In other embodiments, the positioning connectors could be omitted altogether. The upper and lower positioning connectors 180a, 180b are recesses that define an arcuate profile and that are configured to receive two adjacent track connectors 82 which are protrusions defining arcuate profiles. Thus, the upper and lower positioning connectors 180a, 180b are complementary to the track connectors 83, such that the upper and lower positioning connectors 180a, 180b can connect with two track connectors 82. The use of arcuate profile can facilitate cleaning. It is contemplated that the upper and lower positioning connectors 180a, 180b could define other shapes and/or could be protrusions, so long as positioning connectors 180a, 180b are complementary to the track connectors 82 such that an inter-engagement is achieved.

The positioning member 96 is moveable, relative to the outer member 90, between a contracted position (FIGS. 5A and 5B) and an extended position (FIGS. 6A and 6B). When the fixing assembly 54 is connected to the track 52, the movement of the positioning member 96, between the extended and contracted positions is generally perpendicular to the lateral center plane 66 (i.e., generally perpendicular to the longitudinal axis 62). As will be described in greater detail below, when the positioning member 96 is in the extended position, the distal portions 172 are configured to engage the elongate body 60 such that fixing assembly 54 is generally fixed relative to the track 52, whereas when the positioning member 96 is in the contracted position, the distal portions 172 are configured to be disengaged from the elongate body 60 such that the fixing assembly 54 can move relative to the track 52 (parallel to the longitudinal axis 62).

As mentioned above, the fixing assembly 54 is selectively connectable to the track 52.

Referring to FIGS. 6A to 6C, a description of the fixing assembly 54 being connected to the track 52 will now be provided. Although the following passages describe the fixing assembly 54 being connected to the track 52 by the slot 70a, it is understood that in other embodiments, the fixing assembly 54 could be connected to the track 52 by the slot 70b. In other embodiments, one of the slots 70a, 70b could be omitted.

First, the distal portions 172 are received in the slot 70a. In some embodiments, for instance where the upper, intermediate and lower pair of retaining arms 94a, 94b, 94c are laterally fixed relative to the positioning member 96 in the retaining position (i.e., do not retract laterally), the fixing assembly 54 could be connected to the track 54 by sliding the distal portions 172 into the slot 70a from a top thereof the slot 70a, as the top of the slot 70a is open-ended.

In the present embodiment, however, the upper, intermediate and lower pair of retaining arms 94a, 94b, 94c are to be moved to their release position. To this end, the locking member 92 is rotated by inserting a tool such as the hex key (not shown) into the recess 152 and rotating. It is contemplated that in some embodiments, the locking member 92 can be rotated manually, without the use of any tools. The locking member 92 is rotated to the locking member release position (FIG. 4C). Rotation of the locking member 92 results in the position of the upper, intermediate, and lower engaging portions 154a, 154b, 154c relative to, respectively, the upper, intermediate and lower pair of retaining arms 94a, 94b, 94c being modulated. At a certain point, due to the shape of the upper, intermediate, and lower engaging portions 154a, 154b, 154c being generally oval (i.e., due to their variable diameter), rotation is no longer necessary, as the springs 162a, 162b of each the upper, intermediate and lower pair of retaining arms 94a, 94b, 94c bias the upper, intermediate and lower pair of retaining arms 94a, 94b, 94c toward the release position. In other words, when the locking member 92 is at a certain point between the locking member release position and the locking member retaining position, the locking member 92 is guided to the locking member release position by the force applied by the left and right arms 160a, 160b of the upper, intermediate and lower pair of retaining arms 94a, 94b, 94c. Additionally, once the locking member 92 reaches the locking member release position, due to the left and right arms 160a, 160b of the upper, intermediate and lower pair of retaining arms 94a, 94b, 94c compressing the locking member 92, the locking member 92 is generally locked in the locking member release position. It is understood that to rotate the locking member 92 between the locking member release position and the locking member retaining position, the compressive force of the left and right arms 160a, 160b of the upper, intermediate and lower pair of retaining arms 94a, 94b, 94c must be overcome.

When the upper, intermediate and lower pair of retaining arms 94a, 94b, 94c are in their release position shown in FIG. 4C, their left and right arms 160a, 160b are retracted towards the positioning member 96. Thus, the distal portions 172 can be received in the slot 70a, as the left and right arms 160a, 160b of the upper, intermediate and lower pair of retaining arms 94a, 94b, 94c no longer abut the retaining portions 72a, 72b of the elongate body 60.

Therefore, the upper, intermediate and lower pair of retaining arms 94a, 94b, 94c being in the release position enables a movement of the fixing assembly 54 relative to the track 52 in a direction perpendicular to the lateral center plane 66 (i.e., perpendicular to the longitudinal axis 62). The present configuration allows for connecting the fixing assembly 54 to the track 52 at any point along the longitudinal axis 62, which can be convenient, notably for ease and speed of connection and disconnection process of the mobile equipment to the mounting assembly. In other words, there is no need to connect the fixing assembly 54 from a top of the slot 70a as described hereabove, although this is contemplated, and/or there is no need to align the fixing assembly 54 with a feature of the track 52 such as recesses.

Once the distal portions 172 have been received in the slot 70a, the locking member 96 can be rotated again (overcoming the compressive force of the left and right arms 160a, 160b of the upper, intermediate and lower pair of retaining arms 94a, 94b, 94c), for example with the hex key or manually without any tools, until the locking member 92 is in the locking member retaining position. Once the locking member 92 reaches the locking member retaining position, due to the left and right arms 160a, 160b of the upper, intermediate and lower pair of retaining arms 94a, 94b, 94c compressing the locking member 92, the locking member 92 is generally locked in the locking member retaining position. Thus, the upper, intermediate, and lower engaging portions 154a, 154b, 154c engage the upper, intermediate and lower pair of retaining arms 94a, 94b, 94c to move the upper, intermediate and lower pair of retaining arms 94a, 94b, 94c to the retaining position, as shown in FIG. 5C.

Once the upper, intermediate and lower pair of retaining arms 94a, 94b, 94c are in the retaining positions, the upper, intermediate and lower pair of retaining arms 94a, 94b, 94c extend outwardly from the positioning member 96, and engage with the track 52 to retain connection between the fixing assembly 54 and the track 52. More precisely, the body portions 166 of the left and right retaining arms 160a, 160b of each of the upper, intermediate and lower pair of retaining arms 94a, 94b, 94c abut the retaining portions 72a, 72b of the elongate body 60, thereby delimiting movement of the fixing assembly 54 generally perpendicularly away from the lateral center plane 66 (i.e., perpendicularly away from the longitudinal axis 62).

Then, the fixing assembly 54 can be selectively moved between a locked connected mode and an unlocked connected mode. In the locked connected mode, the fixing assembly 54 is generally fixed relative to the track 52, whereas in the unlocked connected mode, the fixing assembly 54 can move relative to the track 52 in a direction generally parallel to the longitudinal axis 62.

Referring to FIGS. 5A to 5C, the unlocked connected mode will first be described. To adjust the fixing assembly 54 to the unlocked connected mode, the left and right actuators 106a, 106b are actuated and moved toward the actuated position. Thus, the left and right actuators 106a, 106b move generally parallel to the lateral center plane 66 (i.e., generally perpendicular to the longitudinal axis 62). The left and right actuators 106a, 106b can move towards the actuated position, so towards one another in the present embodiment, until the front abutting portions 140 thereof abut the outer body 102 of the outer member 90. The left and right actuators 106a, 106b can be actuated and moved towards the actuated position by a single hand, which can, once again, be convenient, notably for ease and speed of connection and disconnection process of the mobile equipment to the mounting assembly.

The left and right actuators 106a, 106b are moveably connected to the proximal portions 170 of the positioning member 96, such that when the left and right actuators 106a, 106b are moved from the non-actuated position to the actuated position, the positioning member 96 moves from the extended position to the contracted position.

More precisely, as the left and right actuators 106a, 106b are moving towards the actuated position, the upper guiding segment 146a of the left actuator 106a is received in the triangular recess 176a of the upper left segment 174a, the lower guiding segment 146b of the left actuator 106a is received in the triangular recess 176c of the lower left segment 174c, the upper guiding segment 146a of the right actuator 106b is received in the triangular recess 176b of the upper right segment 174b, the lower guiding segment 146b of the right actuator 106b is received in the triangular recess 176d of the lower right segment 174d. Additionally, the actuator engaging portion 178a of the upper left segment 174a is received in the upper recess 144a of the left actuator 106a, the actuator engaging portion 178b of the upper right segment 174b is received in the upper recess 144a of the right actuator 106b, the actuator engaging portion 178c of the lower left segment 174c is received in the lower recess 144b of the left actuator 106a, the actuator engaging portion 178d of the lower right segment 174d is received in the lower recess 144b of the right actuator 106b. As a result of this engagement and the present configuration, as the left and right actuators 106a, 106b move generally parallel to the lateral center plane 66 towards one another other, the positioning member 96 moves generally perpendicularly away from the lateral center plane 66. The movement of the positioning member 96 and relative to the actuators 106a, 106b is guided, in part, by the shapes of the upper and lower recesses 144a, 144b, the upper and lower guiding segments 146a, 146b, the triangular recesses 176a, 176b, 176c, 176d and the actuator engaging portions 178a, 178b, 178c, 178d.

When the left and right actuators 106a, 106b are in the actuated position, and the positioning member 96 is in the contracted position, the distal portions 172 does not engage the elongate body 60 such that the fixing assembly 54 can slide in the slot 70a (i.e., can move parallel to the longitudinal axis 62) to position the fixing assembly 54 at a desired height. The fixing assembly 54 can be slid until the upper positioning connector 180a is aligned with one of the track connectors 82 and the lower positioning connector 180a is aligned with another one of the track connectors 82. In some embodiments, the upper and lower positioning connectors 180a, 180b could be connected to two adjacent track connectors 82.

Turning now to FIGS. 4A to 4C, since the left and right actuators 106a, 106b are biased toward the non-actuated position, releasing the left and right actuators 106a, 106b results in them moving back towards their non-actuated positions and the positioning member 96 moving towards the extended position (i.e., generally perpendicular to the lateral center plane 66 and the longitudinal axis 66 and toward the track 52).

When the left and right actuators 106a, 106b are released while the upper positioning connector 180a is aligned with one of the track connectors 82 and the lower positioning connector 180a is aligned with another one of the track connectors 82, the positioning member 96 moves toward the extended position such that the upper and lower positioning connectors 180a, 180b connect with the track connectors 82, thereby locking the fixing assembly 54 relative to the track 52. Thus, the fixing assembly 54 cannot move parallel to the longitudinal axis 62. It is contemplated that in some embodiments, where the track and positioning connectors are omitted, the distal portions 172 could engage the elongate body, and friction therebetween could lock the fixing assembly 54 relative to the track 52.

To remove the fixing assembly 54 from the track 52, as described hereabove, the locking member 92 can be rotated until the upper, intermediate and lower pair of retaining members 94a, 94b, 94c are in the release position, therefore no longer abutting the retaining portions 72a, 72b. As a result, the fixing assembly 54 can be moved perpendicularly away from the track 52 (i.e., perpendicularly away from the track 52).

Furthermore, it is understood that the mobile equipment can be connected to the outer member 90 of the fixing assembly 54 before or after the fixing assembly 54 is connected to the track 52.

It is contemplated that in some embodiments, there could be two or more fixing assemblies 54 selectively connected to the track 52, for instance to connect two different components of mobile equipment to the track 52.

Modifications and improvements to the above-described embodiments of the present invention may become apparent to those skilled in the art. The foregoing description is intended to be exemplary rather than limiting. The scope of the present invention is therefore intended to be limited solely by the appended claims.

What is claimed is:

1. A mounting assembly comprising:
a track comprising:
an elongate body having a longitudinal axis,
a slot defined in the elongate body and extending longitudinally;
a fixing assembly which can be selectively connected and disconnected from the track in a direction perpendicular to the longitudinal axis, and wherein when connected to the track, the fixing assembly is moveable longitudinally in the slot in an unlocked connected mode, and is lockable in the slot in a locked connected mode, the fixing assembly comprising:
an outer member;
a positioning member operatively connected to the outer member, the positioning member having a distal portion receivable in the slot, wherein the positioning member is movable, relative to the outer member, between:
an extended position, in the locked connected mode, in which the distal portion is engaged with the elongate body of the track when the distal portion of the positioning member is received in the slot for longitudinally fixing the fixing assembly relative to the track, and
a contracted position, in the unlocked connected mode, in which the distal portion is disengaged with the elongate body of the track when the distal portion of the positioning member is received in the slot,
a retaining member selectively extendable from the distal portion of the positioning member, the retaining member being movable between:
a retaining position in which, when the distal portion of the positioning member is received in the slot, the retaining member engages with the track for retaining connection between the fixing assembly and the track, and
a release position in which the fixing assembly can be disconnected from the track.

2. The mounting assembly of claim 1, wherein:
the retaining member comprises a pair of arms such that, when the distal portion of the positioning member is received in the slot:
in the retaining position, each arm of the pair of arms extends laterally outwardly from the distal portion of the positioning member and delimits movement of the fixing assembly perpendicularly away from the longitudinal axis of the track to retain the connection between the fixing assembly and the track, and
in the release position, each arm of the pair of arms is retracted towards the distal portion of the positioning member to permit movement of the fixing assembly perpendicularly away from the longitudinal axis of the track to disconnect the fixing assembly from the track.

3. The mounting assembly of claim 2, wherein each arm of the pair of arms extends through a wall of the distal portion of the positioning member, the retaining member further comprising a locking member disposed between the pair of arms, the locking member configured to selectively modulate a separation distance between the pair of arms, such that the separation distance of the pair of arms when the pair of arms are in the retaining position is larger than the separation distance when the pair of arms is in the release position.

4. The mounting assembly of claim 3, wherein the locking member comprises a shaft extending parallel to the longitudinal axis when the distal portion of the positioning member is received in the slot, the shaft having a cross-sectional profile with a variable diameter, such that the separation distance between the pair of arms can be modulated by moving the shaft.

5. The mounting assembly of claim 4, wherein the locking member has a limiting portion for limiting movement of the shaft.

6. The mounting assembly of claim 4 or 5, wherein the shaft is rotatable and the separation distance between the arms of the pair of arms can be modulated by rotating the shaft.

7. The mounting assembly of claim 4 or 5, wherein the shaft is moveable along a longitudinal axis of the shaft and the separation distance between the arms of the pair of arms can be modulated by translating the shaft along the longitudinal axis.

8. The mounting assembly of any one of claims 3 to 7, wherein the pair of arms are resiliently biased towards each other by a resilient member.

9. The mounting assembly of any one of claims 3 to 8, wherein the pair of arms is a first pair of arms, and the retaining member comprises a second pair of arms spaced longitudinally from the first pair of arms.

10. The mounting assembly of claim 9, wherein the retaining member comprises a third pair of arms, and the first pair of arms, the second pair of arms and the third pair of arms are spaced longitudinally from one another.

11. The mounting assembly of any one of claims 3 to 10, wherein the outer member includes an inner body, and the locking member is received at least partially within the inner body.

12. The mounting assembly of any one of claims 1 to 11, wherein the outer member includes an actuator which is moveably connected to a proximal portion of the positioning member, the actuator being moveable between an actuated position and a non-actuated position to selectively move the positioning member between the contracted position and the extended position, respectively.

13. The mounting assembly of claim 12, wherein the actuator is biased toward the non-actuated position.

14. The mounting assembly of claim 13, wherein the actuator is biased by a resilient member.

15. The mounting assembly of any one of claims 12 to 14, wherein a direction of movement of the actuator between the actuated position and the non-actuated position is perpendicular to a direction of movement of the positioning member between the contracted position and the extended position.

16. The mounting assembly of any one of claims 1 to 15, wherein the positioning member moves between the extended and contracted positions perpendicularly to the longitudinal axis, along a positioning plane containing the longitudinal axis.

17. The mounting assembly of any one of claims 1 to 16, wherein:
the positioning member includes a first connector;
the track includes a plurality of second connectors longitudinally spaced from one another, and
wherein the first connector connects to at least one of the plurality of second connectors when the fixing assembly is in the locked connected mode and the positioning member is in the extended position.

18. The mounting assembly of claim 17, wherein each one of the plurality of second connectors comprises a protrusion, and the first connector is a recess configured to receive the at least one of the plurality of second connector.

19. The mounting assembly of claim 17 or 18, wherein the first connector and the plurality of second connectors each have an arcuate profile.

20. The mounting assembly of any one of claims 17 to 19, wherein the first connector is an upper first connector and a lower first connector, and wherein the upper first connector is configured to connect to one of the plurality of second connectors and the lower first connector is configured to connect to an other one of the plurality of second connectors.

21. The mounting assembly of any one of claims 17 to 20, wherein the track includes a first insert having the plurality of second connectors, the first insert being replaceable with a second insert.

22. The mounting assembly of any one of claims 1 to 21, wherein:
the slot is a first slot on a first side of the elongate body;
the elongate body has a second side opposite to the first side, the second side defining a second slot extending longitudinally along the second side, the second slot having a plurality of third connectors spaced from one another along the second side, the third connectors being configured to connect to the first connector, and
the fixing assembly is selectively connectable to the track at either one of the first and second sides.

23. The mounting assembly of claim 22, wherein the elongate body further has:
a third side defining a third slot extending longitudinally along the third side, the third slot having a plurality of fourth connectors spaced from one another along the third side, the fourth connectors being configured to connect to the first connector;
a fourth side opposite to the third side, the fourth side defining a fourth slot extending longitudinally along the fourth side, the fourth slot having a plurality of fifth connectors spaced from one another along the fourth side, the fifth connectors being configured to connect to the first connector, and
the fixing assembly is selectively connectable to the track at either one of the first, second, third and fourth sides.

24. The mounting assembly of any one of claims 1 to 23, wherein the fixing assembly is configured to connect mobile equipment to the track.

25. A mounting assembly comprising:
a track comprising:
an elongate body having a longitudinal axis,
a slot defined in the elongate body and extending longitudinally;
a fixing assembly selectively connectable and disconnectable from the track in a direction perpendicular to the longitudinal axis, and wherein when connected to the track, the fixing assembly is moveable longitudinally in the slot, the fixing assembly comprising:
an outer member;
a positioning member operatively connected to the outer member, the positioning member being at least partially receivable in the slot and being engageable to the elongate body for longitudinally fixing the fixing assembly relative to the track;
a retaining member selectively laterally extendable from the positioning member, the retaining member being movable between:
a retaining position in which, when the positioning member is at least partially received in the slot, the retaining member engages with the track to retain connection between the fixing assembly and the track, and
a release position in which the fixing assembly can be disconnected from the track.

26. The mounting assembly of claim 25, wherein:
the retaining member comprises a pair of arms such that, when the positioning member is at least partially received in the slot:
in the retaining position, each arm of the pair of arms extends laterally outwardly from the positioning member and delimits movement of the fixing assembly perpendicularly away from the longitudinal axis of the track to retain the connection between the fixing assembly and the track, and
in the release position, each arm of the pair of arms is retracted towards the positioning member to permit movement of the fixing assembly perpendicularly away from the longitudinal axis of the track to disconnect the positioning member from the track.

27. The mounting assembly of claim 26, wherein each arm of the pair of arms extends through a wall of the positioning member, the retaining member further comprising a locking member disposed between the pair of arms, the locking member configured to selectively modulate a separation distance between the pair of arms, such that the separation distance of the pair of arms when the pair of arms are in the retaining position is larger than the separation distance when the pair of arms is in the release position.

28. The mounting assembly of claim 27, wherein the locking member comprises a shaft extending parallel to the longitudinal axis when the positioning member is at least partially received in the slot, the shaft having a cross-sectional profile with a variable diameter, such that the separation distance between the pair of arms can be modulated by moving the shaft.

29. The mounting assembly of claim 28, wherein the locking member has a limiting portion for limiting movement of the shaft.

30. The mounting assembly of claim 28 or 29, wherein the shaft is rotatable and the separation distance between the arms of the pair of arms can be modulated by rotating the shaft.

31. The mounting assembly of claim 28 or 29, wherein the shaft is moveable along a longitudinal axis of the shaft and the separation distance between the arms of the pair of arms can be modulated by translating the shaft along the longitudinal axis.

32. The mounting assembly of any one of claims 27 to 31, wherein the pair of arms are resiliently biased towards each other by a resilient member.

33. The mounting assembly of any one of claims 27 to 32, wherein the pair of arms is a first pair of arms, and the retaining member comprises a second pair of arms spaced longitudinally from the first pair of arms.

34. The mounting assembly of claim 33, wherein the retaining member comprises a third pair of arms, and the first pair of arms, the second pair of arms and the third pair of arms are spaced longitudinally from one another.

35. The mounting assembly of any one of claims 27 to 34, wherein the outer member includes an inner body, and the locking member is received at least partially within the inner body.

36. The mounting assembly of any one of claims 25 to 35, wherein the fixing assembly is configured to connect mobile equipment to the track.

37. The mounting assembly of any one of claims 25 to 36, wherein:
the slot is a first slot on a first side of the elongate body;
the elongate body has a second side opposite to the first side, the second side defining a second slot extending longitudinally along the second side, the second slot having a plurality of third connectors spaced from one another along the second side, the third connectors being configured to connect to the first connector, and
the fixing assembly is selectively connectable to the track at either one of the first and second sides.

38. The mounting assembly of claim 37, wherein the elongate body further has:
a third side defining a third slot extending longitudinally along the third side, the third slot having a plurality of fourth connectors spaced from one another along the third side, the fourth connectors being configured to connect to the first connector;
a fourth side opposite to the third side, the fourth side defining a fourth slot extending longitudinally along the fourth side, the fourth slot having a plurality of fifth connectors spaced from one another along the fourth side, the fifth connectors being configured to connect to the first connector, and
the fixing assembly is selectively connectable to the track at either one of the first, second, third and fourth sides.

39. A mounting assembly comprising:
a track comprising:
an elongate body having a longitudinal axis,
a slot defined in the elongate body and extending longitudinally;
a fixing assembly connectable to the track, and the fixing assembly comprising:
an outer member including an actuator;
a positioning member operatively connected to the outer member, the positioning member having a proximal portion and a distal portion receivable in the slot, and wherein the positioning member is movable, relative to the outer member, between:
an extended position, in which when the distal portion is received in the slot, the distal portion engages the elongate body of the track for longitudinally fixing the fixing assembly relative to the track, and
a contracted position, in which when the distal portion is received in the slot, the distal portion is disengaged from the elongate body of the track for permitting longitudinal movement of the fixing assembly relative to the track,
wherein the actuator is moveably connected to the proximal portion of the positioning member, the actuator being selectively moveable between:
an actuated position to move the positioning member to the contracted position, and
a non-actuated position in which the positioning member is in the extended position.

40. The mounting assembly of claim 39, wherein the actuator is biased toward the non-actuated position.

41. The mounting assembly of claim 40, wherein the actuator is biased by a resilient member.

42. The mounting assembly of any one of claims 39 to 41, wherein a direction of movement of the actuator between the actuated position and the non-actuated position is perpendicular to a direction of movement of the positioning member between the contracted position and the extended position.

43. The mounting assembly of any one of claims 39 to 42, wherein the positioning member moves between the extended and contracted positions perpendicularly to the longitudinal axis, along a positioning plane containing the longitudinal axis.

44. The mounting assembly of any one of claims 39 to 43, wherein:
the positioning member includes a first connector on the distal portion; and
the elongate track includes a plurality of second connectors longitudinally spaced from one another, and
wherein the first connector connects to one of the plurality of second connectors when the positioning member is in the extended position.

45. The mounting assembly of claim 44, wherein each one of the plurality of second connectors comprises a protrusion, and the first connector is a recess configured to receive one of the plurality of second connector.

46. The mounting assembly of any one of claim 44 or 45, wherein the first connector and the plurality of second connectors each have an arcuate profile.

47. The mounting assembly of any one of claims 44 to 46, wherein the first connector is an upper first connector and a lower first connector, and wherein the upper first connector is configured to connect to one of the plurality of second connectors and the lower first connector is configured to connect to an other one of the plurality of second connectors.

48. The mounting assembly of any one of claims 44 to 47, wherein the track includes a first insert having the plurality of second connectors, the first insert being replaceable with a second insert.

49. The mounting assembly of any one of claims 44 to 48, wherein:
the slot is a first slot on a first side of the elongate body;
the elongate body has a second side opposite to the first side, the second side defining a second slot extending longitudinally along the second side, the second slot having a plurality of third connectors spaced from one another along the second side, the third connectors being configured to connect to the first connector, and
the fixing assembly is selectively connectable to the track at either one of the first and second sides.

50. The mounting assembly of claim 49, wherein the elongate body further has:
a third side defining a third slot extending longitudinally along the third side, the third slot having a plurality of fourth connectors spaced from one another along the third side, the fourth connectors being configured to connect to the first connector;
a fourth side opposite to the third side, the fourth side defining a fourth slot extending longitudinally along the fourth side, the fourth slot having a plurality of fifth connectors spaced from one another along the fourth side, the fifth connectors being configured to connect to the first connector, and
the fixing assembly is selectively connectable to the track at either one of the first, second, third and fourth sides.

51. The mounting assembly of any one of claims 44 to 50, wherein the fixing assembly is configured to connect mobile equipment to the track.

52. A fixing assembly selectively connectable to a track, the fixing assembly comprising:
an outer member;
a positioning member operatively connected to the outer member, the positioning member having a distal portion configured to be receivable in the slot, and the positioning member being movable between: an extended position and a contracted position, a distance from the outer member to the distal portion being greater when the positioning member is in the extended position than when the positioning member is in the contracted position;
a retaining member selectively extendable from the distal portion of the positioning member, the retaining member being movable between a retaining position in which the retaining member extends laterally from the distal portion of the positioning member and a release position in which the retaining member is retracted towards the distal portion of the positioning member.

53. A fixing assembly selectively connectable to a track, the fixing assembly comprising:
an outer member;
a positioning member operatively connected to the outer member, the positioning member having at least one positioning connector configured to connect to a connector of the track, wherein the positioning member is movable between an extended position and a contracted position,
a retaining member operatively connected to the positioning member, the retaining member being movable between a retaining position and a release position; and
a locking member operatively connected to the positioning member and engaged to the retaining member, the locking member being adjustable between an unlocked position and a locked position, and
in the unlocked position, the locking member engages the retaining member to move the retaining member to the release position; and
in the locked position, the locking member engages the retaining member to move the retaining member to the retaining position.
